(19) United States
(12) Patent Application Publication
(10) Pub. No.: US 2009/0317918 A1
(43) Pub. Date: Dec. 24, 2009

(54) METHOD OF HEATING LIQUID MEDIUM USING MICROWAVES AND ANIONS

(75) Inventors: Kyu-youn Hwang, Yongin-si (KR); Joon-ho Kim, Seongnam-si (KR); Koong-kak Nam, Seoul (KR); Won-seok Chung, Hwaseong-si (KR); Hee-kyun Lim, Hwaseong-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/553,402

(22) Filed: Sep. 3, 2009

(30) Foreign Application Priority Data
Mar. 9, 2008 (KR) 10-2008-0086711

(51) Int. Cl. G01N 1/00; G01N 33/48; C12M 1/00

(52) U.S. Cl. 436/175; 422/68.1; 435/287.1

(57) ABSTRACT

Provided is a method comprising adding anions having a high charge density to a liquid medium; the liquid medium comprising molecules that hydrogen bond with one another; the anions interacting with the molecules of the liquid medium with a force that is stronger than the forces that produce hydrogen bonding between the molecules of the medium; and heating the liquid medium by irradiating it with microwaves.

### CROSS-REFERENCE TO RELATED APPLICATIONS

This application claims priority to Korean Patent Application No. 10-2008-0086711, filed on Sep. 3, 2008 and all benefits accruing therefrom under 35 U.S.C. § 119, the contents of which in its entirety are herein incorporated by reference.

### BACKGROUND

1. Field

One or more embodiments relate to a method of heating a liquid medium using microwaves, and more particularly, to a method of heating the liquid medium comprising anions by using microwaves.

2. Description of the Related Art

Analysis on clinical or environmental samples is performed via a series of biochemical, chemical, and mechanical processes. Recently, a technology that involves reacting a biological sample on a chip or disk has been used for analyzing and detecting desired biomolecules. This technique is regarded as having tremendous potential for analyzing different types of chemicals and reactions.

In addition, a nucleic acid based molecule diagnosing method has excellent accuracy and sensitivity, and thus is widely used in pharmacogenomics or in diagnosing infectious diseases such as cancers.

However, in order to diagnose a desired molecule, the target molecule to be detected and analyzed is to be separated from a biological sample and then purified in a pretreatment process, for example, an amplification process, such as a polymerase chain reaction (“PCR”). An important process for extracting the biomolecule includes lysing a target cell and purifying biomolecules derived therefrom.

In order to accomplish the separation, a molecule extraction kit manufactured by QIAGEN or MOLZYM is used to sequentially perform a series of operations, such as a cell lysing process, a deoxyribonucleic acid (“DNA”) capturing process, a washing process, and a DNA discharging process, while performing a separate heating process during a corresponding operation. However, the molecular recovery rate is very low and it is difficult to control the various operations listed above.

The various operations listed above may be automatically performed in a microfluidic apparatus. The microfluidic apparatus is also heated in order to automatically extract a biomolecule. The heating is also desirable when lysing a cell. This heating is advantageous when nucleic acid is heated during elution after binding the nucleic acid to a solid material. Heating increases nucleic acid separating efficiency.

Heating is generally accomplished by resistive heating where an electrical current is passed through the body that is to be heated. However when the resistive heating method is used in a microfluidic apparatus that contains a plurality of microfluidic parts and structures, the method of controlling the heat in different parts of the microfluidic structure becomes complicated. To effectively control heating, a heater may be built inside the microfluidic apparatus or may be mechanically constructed to contact the system from the outside. In addition, since microfluidic apparatus are mostly made out of plastic, heating efficiency decreases and the amount of time used for heating the parts of the apparatus to a desired temperature increases.

### SUMMARY

One or more embodiments include a method of heating a liquid medium; the liquid medium including anions, which have a high charge density; the anions accelerating the polarization of the molecules in the liquid medium, which can reduce heating time by increasing the heating rate.

One or more embodiments may include a method of heating a liquid medium using microwaves; the method including adding anions having high charge density to a liquid medium containing a hydrogen bond between liquid medium molecules; the anions having a high charge density that accelerates the polarization of the molecules of the medium; and heating the liquid medium by irradiating it with microwaves.

One or more embodiments include a biological analysis device, which includes a plurality of chambers containing a liquid medium containing a hydrogen bond between the molecules of the liquid medium; the liquid medium further including anions having a high charge density; the biological analysis device including the liquid medium including the anions in some chambers to be heated, and selectively heating a desired chamber.

One or more embodiments include a liquid medium including anions having high charge density that accelerates polarization of molecules of the medium; and heating the liquid medium including the anions using microwaves.

Additional aspects will be set forth in part in the description which follows and, in part, will be apparent from the description, or may be learned by practice of the invention.

To achieve the above and/or other aspects, one or more embodiments may include a method of heating a liquid medium including anions, the method including adding anions having high charge density to a liquid medium containing a hydrogen bond between the molecules of the liquid medium; the anions having a high charge density that accelerates the polarization of molecules of the liquid medium; and heating the liquid medium by irradiating it with microwaves. The anions have a high charge density that accelerates the polarization of molecules of the liquid medium. The anions interact with the molecules of the liquid medium with a force that is stronger than the force between hydrogen bonds between the medium molecules.

One or more embodiments may include a method of heating a liquid medium including anions, the method including adding anions having a high charge density to a liquid medium; the liquid medium including molecules that hydrogen bond with one another; the anions interacting with the molecules of the liquid medium with a force that is stronger than the force exerted by the hydrogen bonds that exist between the molecules of the liquid medium; and heating the liquid medium including the anions by irradiating it with microwaves.

The liquid medium may be a material having hydrogen bonds between the molecules, and may include any one of water, an aqueous solution, a buffer solution, and mixtures thereof. The liquid medium can be a microfluid that occupies a volume of about several nanoliters to hundreds of microliters. The liquid medium may display dielectric properties. The molecules of the liquid medium may display electrical properties in addition to having a hydrogen bond between the molecules.

The anions having high charge density may interact with the molecules of the liquid medium forming a dielectric substance with a force that is stronger than the force exerted between molecules that are only hydrogen bonded with one another. The anions having a high charge density may be citric acid ions (citrate³⁻), sulfate ions (SO₄²⁻), hydrogen sulfate ions (HSO₄⁻), phosphate ions (PO₄³⁻), hydrogen phosphate ions (HPO₄²⁻), carbonate ions (CO₃²⁻), hydrogen carbonate ions (HCO₃⁻), or a combination comprising at least one of the foregoing ions.

The anions having a high charge density may be added in a concentration of about 10 millimolar (“mM”) to 10 molar (“M”), and may be about 100 mM to about 3 M.

The microwave may have a frequency of about 300 megahertz (“MHz”) to about 300 gigahertz (“GHz”), about 300 MHz to about 30 GHz, and about 1 GHz to about 30 GHz.

To achieve the above and/or other aspects, one or more embodiments may include a method of heating a liquid medium including preparing a chamber for a biological analysis device; the chamber containing a liquid medium where the molecules are hydrogen bonded to one another; adding anions having high charge density to the chamber, the anions interacting with the molecules of the liquid medium; the anions interact with the molecules of the liquid medium with a force that is stronger than a hydrogen bond force between the molecules of the liquid medium; and heating the liquid medium including the anions by irradiating it with microwaves.

The chamber may be any one of a lysis solution chamber that lyses cells in a biological sample, and an elution solution chamber that elutes a biomolecule from a solid material to which the biomolecule is bound.

The anions having high charge density and the anions interact with the molecules of the liquid medium with a force that is stronger than a hydrogen bond force between the molecules of the liquid medium, so as to increase a heating rate of the liquid medium. The anions having a high charge density may be one of citric acid ions (citrate³⁻), sulfate ions (SO₄²⁻), hydrogen sulfate ions (HSO₄⁻), phosphate ions (PO₄³⁻), hydrogen phosphate ions (HPO₄²⁻), carbonate ions (CO₃²⁻), hydrogen carbonate ions (HCO₃⁻), or a combination comprising at least one of the foregoing ions.

The anions having high charge density may be added in a concentration about 10 mM to about 10 M, or about 100 mM to about 3 M.

To achieve the above and/or other aspects, one or more embodiments may include a biological analysis device containing anions, the biological analysis device including a plurality of chambers for processing, removing, or reacting a sample; a liquid medium contained in the plurality of chambers; the liquid medium including molecules that hydrogen bond with one another; and anions having a high charge density, wherein the anions are added to some of the plurality of chambers and interact with the molecules of the liquid medium with a force that is stronger than a hydrogen bond force between the molecules of the liquid medium.

The medium may include any one of water, an aqueous solution, organic solvent, a buffer solution, and mixtures comprising at least one of the foregoing. The liquid may be a microfluid that occupies a volume of about several nanoliters to hundreds of microliters.

The biological analysis device may be an apparatus for detecting or analyzing a biomolecule from a biological sample, such as a microfluidic device for detecting or analyzing a biomolecule from a biological sample, a microfluidic cartridge, a lab-on-a chip, or a lab-on-a disc.

The biological sample may include any one of a cell suspension including microorganisms and blood, urine, or saliva of a human, and the biomolecule may include any one of nucleic acid, protein, peptide, antibody, and hormone. The nucleic acid may include any one of DNA and ribonucleic acid (“RNA”).

The plurality of chambers may include a lysis solution chamber for storing a solution which lyses cells in a biological sample; a binding solution chamber for storing a solution which binds biomolecules discharged from the lysed cells to a solid material; a washing solution chamber for storing a solution which removes biomolecules that are not bound to the solid material by washing the solid material; and an elution solution chamber for storing a solution which elutes the biomolecules from the solid material to which the biomolecules are bound.

The anions having high charge density may be added only to those chambers among the plurality of chambers where heating of the liquid medium is desired. The chambers that are to be heated may be a lysis solution chamber and an elution solution chamber.

The biological analysis device may further include anions having low charge density, which are added to chambers that are not to be heated from among the plurality of chambers. The chambers that are not to be heated may be a binding solution chamber and a washing solution chamber. The anions having low charge density may include at least one of acetic acid ions (acetate), chloride ions (Cl⁻), nitrate ions (NO₃⁻), bromide ions (Br⁻), chloric ions (ClO₃⁻), perchloric ions (ClO₄⁻), iodides (I⁻), and thiocyanate ions (SCN⁻), or a combination comprising at least one of the foregoing low charge density anions. The term “low charge density” may be considered in terms of a relationship with the medium molecules. Accordingly, the anions having low charge density may have entropy of hydration 6.3 kilojoules per mole (“KJ/mol”) or less.

To achieve the above and/or other aspects, one or more embodiments may include a liquid medium for heating using microwaves, the liquid medium including molecules of the liquid medium that undergo hydrogen bonding between the molecules of the liquid medium; and anions having high charge density, the anions interacting with the medium molecules such that the force between the anions and the molecules of the liquid medium is greater than the forces due to the hydrogen bonding. The medium may include any one of water, an aqueous solution, organic solvent, and a buffer solution.

### BRIEF DESCRIPTION OF THE DRAWINGS

The above and other aspects, advantages and features of the invention will become more apparent by describing in further detail exemplary embodiments thereof with reference to the attached drawings, in which:

FIG. 1 is a conceptual diagram for explaining the principle of dielectric heating;

FIG. 2 is an exemplary frontal schematic diagram illustrating a microfluidic device;

FIG. 3 is a graph showing average heating temperatures according to types of anions in Experimental Example 1;

FIG. 4 is a graph showing average heating temperatures according to anion charge densities in Experimental Example 2; and

FIG. 5 is a graph showing average heating temperatures according to concentrations of anions in Experimental Example 3.

### DETAILED DESCRIPTION

Reference will now be made in detail to embodiments, examples of which are illustrated in the accompanying drawings. In this regard, the present embodiments may have different forms and should not be construed as being limited to the descriptions set forth herein. Accordingly, the embodiments are merely described below, by referring to the figures, to explain aspects of the present description.

The invention now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

The transition phrases such as including or comprising may be replaced with the transition phrases “consisting of” and “consisting essentially of”, as and when the Applicants desire.

When detailed research was conducted about non-contact type methods of heating a liquid medium containing hydrogen bonds between the molecules of the medium, it was found that the heating rate increases when anions having a high charge density are added to the medium, the anions accelerating polarization of the medium molecules, for example, polar molecules.

In one embodiment, the liquid medium may be heated by adding anions having high charge density that accelerates the polarization of the molecules in the liquid medium. The liquid medium including the anions is then heated by irradiating it with microwaves. By using this method, the heating rate of the liquid medium may be increased significantly. Here, the anions may interact with the molecules of the liquid medium with a force that is stronger than the forces that produce hydrogen bonding between the molecules of the medium.

A method according to an embodiment includes adding anions having high charge density to the liquid medium. The molecules of the liquid medium can undergo hydrogen bonding with one another. The anions interact with the molecules of the liquid medium with a force that is stronger than the forces that produce hydrogen bonding between the molecules of the medium.

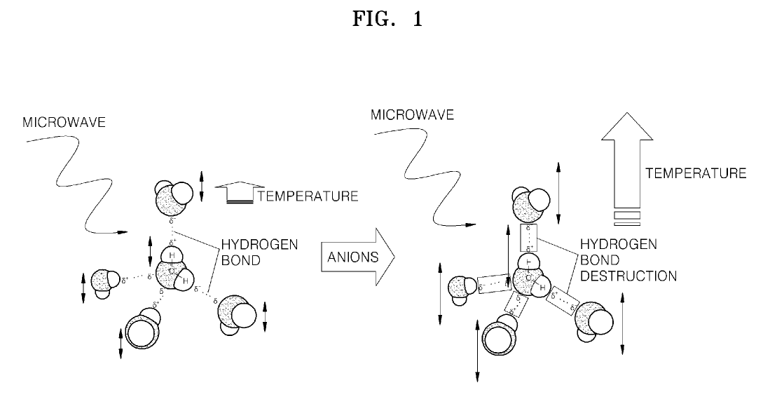

FIG. 1 is a conceptual diagram for explaining the principles of heating molecules with radiation including microwaves. Referring to FIG. 1, when microwaves are applied to a liquid medium containing the anions having a high charge density, the anions accelerate polarization of the medium molecules, thereby contributing to breakage of the hydrogen bond between the medium molecules. Accordingly, the heating rate of the liquid medium is increased.

In one embodiment, heating is conducted by irradiating the liquid medium with high-frequency microwaves. One or more embodiments include a method of heating an object via an indirect, non-contacting method, by heating a medium or an object included in the liquid medium.

For example, when the medium is water, microwaves having a frequency similar to the resonance frequency of water are applied to heat the liquid medium. The water medium molecules undergo resonance with the microwaves, thereby absorbing wave energy. Consequently, the molecules of the liquid medium rotate and thus generate frictional heat. As a result, a heating effect is realized, which increases the temperature of the liquid medium.

In other words, when an electrostatic field (direct current) is applied to polar water molecules, hydrogen having a positive charge is aligned towards a cathode, and oxygen having a negative charge is aligned towards an anode. Hence, when an alternating current (“AC”) electric field is applied (i.e., wherein a direction of the applied electric field changes instantaneously), the water molecules that were aligned are realigned while rotating according to the direction of the electric field. During such realignment, the water molecules produce friction, thereby generating heat.

The microwaves used in embodiments may have similar frequencies to the frequency used in dielectric heating of a sample. The frequency of the microwaves may be about 300 MHz to about 300 GHz, about 300 MHz to about 30 GHz, or about 1 GHz to about 30 GHz.

A device for generating the microwaves is not limited, and any well-known device may be used, such as an electron tube, a klystron, a magnetron, a waveguide, or a laser.

According to an embodiment, the medium may be water, an aqueous solution, organic solvent, a buffer solution, or a combination comprising at least one of the foregoing fluids. The medium may be present in a “microfluid” volume having a volume of several nano liters to hundreds of micro liters. The liquid medium is one where the molecules can form a hydrogen bond with one another. For example, the molecule may contain a hydrogen atom bonded to an atom selected from the group consisting of nitrogen, oxygen and fluorine. The molecule includes water, alcohols such as methanol, ethanol, propanol, and the like; ammonia, methyl amine, hydrogen fluoride (“HF”), and the like.

When the liquid medium is an organic material, the liquid medium may be heated via jacket type or bath type heating by heating a container containing the liquid medium, while the container is being surrounded by a solution containing anions having high charge density.

Anions having a high charge density are added to a liquid medium in order to increase the heating rate.

The anions having a high charge density interact with the molecules of the liquid medium with a force that is stronger than the forces that produce hydrogen bonding between the molecules of the medium or may be anions that can weaken or destroy the hydrogen bonds between the medium molecules.

In other words, the concept of “high charge density” may be considered in terms of a relationship between the molecules of the liquid medium. Accordingly, the anions having a high charge density may have entropy of hydration of greater than or equal to about 6.3 KJ/mol.

As described above, when the anions having high charge density are added to the liquid medium, such as, for example, a dielectric substance, the anions form a stronger interaction with the molecules of the liquid medium than the hydrogen bonding that occurs between the medium molecules, i.e., the anions form an attraction force between anion-positive dipoles. Accordingly, a three-dimensional structure generated by the hydrogen bond is weakened or destroyed.

For example, when the medium is water, the strength of the hydrogen bonds between the water molecules is 6.3 KJ/mol, whereas an energy of hydrating the anions having high charge density, such as sulfate ions (SO₄²⁻), in water is 46 KJ/mol. Such energies generally increase as the charge density increases.

According to an embodiment, the anions having high charge density may be one of citric acid ions (citrate³⁻), sulfate ions (SO₄²⁻), hydrogen sulfate ions (HSO₄⁻), phosphate ions (PO₄³⁻), hydrogen phosphate ions (HPO₄²⁻), carbonate ions (CO₃²⁻), hydrogen carbonate ions (HCO₃⁻), or a combination comprising at least one of the foregoing ions. Examples of suitable anions are citric acid ions (citrate³⁻) or sulfate ions (SO₄²⁻).

The anions may be added in a form of a salt and an ionic compound including the anions. Cations for forming the salt may be H⁺, Na⁺, NH₄⁺, K⁺, Li⁺, Mg²⁺, Ca²⁺, or the like, and the ionic compound may be sodium dodecyl sulfate (“SDS”), sodium octyl sulfate, or the like, or a combination comprising at least one of the foregoing ionic compounds. The cations and the ionic compound are not limited thereto.

As such, the heating method using the microwaves and the anions may be variously used in order to heat a material with or without direct contact with the microwave generator, like heating an organic solvent in a bath or drying moisture from a substance.

A heating method according to another embodiment includes a method of heating a fluid in a biological analysis device by using the heating method according to the previous embodiment.

In detail, the heating method according to the current embodiment includes preparing a chamber for a biological analysis device, wherein the chamber contains a liquid medium whose molecules can undergo hydrogen bonding; adding anions having high charge density to the chamber, wherein the anions interact with molecules of the liquid medium. The liquid medium including the anions is then heated using microwaves. As noted above, the anions interact with the molecules of the liquid medium with a force that is stronger than the forces that produce hydrogen bonding between the molecules of the liquid medium.

The biological analysis device is not limited as long as it contains a liquid medium containing a hydrogen bond between the molecules of the liquid medium. The biological analysis device is a device for diagnosing or monitoring a biological sample or a device for analyzing and detecting a biomolecule. According to an embodiment, the biological analysis device may be a microfluidic device including a microfluidic structure containing a microfluid having a volume of about several nano liters to hundreds of micro liters, a microfluidic cartridge, a microchip, a lab-on-a chip, or a lab-on-a disc.

Also, the amount and type of the biological sample are not limited as long as the biological sample includes biomolecules. The biological sample may be a cell suspension including microorganisms, blood, urine, saliva of a living being including human beings, or the like. The biomolecule may vary according to the purpose of the analysis, and may be a nucleic acid, i.e., DNA or RNA, protein, a peptide, an enzyme, an antibody, a nucleotide, an oligonucleotide, an antigen, an enzyme substrate, an enzyme inhibitor, a transition state analog of an enzyme substrate or a combination thereof.

The biological analysis device using microfluid may perform a preprocess operation on a biological sample. Examples of such preprocess operations are amplification processes, a polymerase chain reaction (“PCR”) amplifying operation, an electrophoresis operation, a sensing operation, or combination of such operations. Here, the preprocess operation determines completion of a molecule diagnosis. The biomolecules are separated during the preprocess operation. Examples of suitable separations are those that separate red blood cells and leukocytes from blood, heterogeneous material separation that separates DNA, RNA, or protein from various materials in a cytoplasm, and congener separation that separates a certain DNA from DNAs having various lengths.

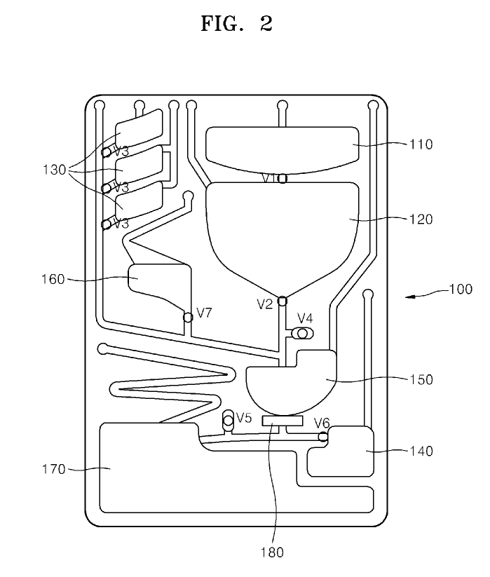

FIG. 2 is a frontal schematic diagram illustrating a microfluidic device 100. Referring to FIG. 2, the microfluidic device 100 analyzes a small amount of sample, and includes micro components for processing a fluid. Examples of the micro components include a channel, a pump, a plurality of micro-reaction chambers, an electrophoresis module, a micro channel, a fluid storage unit, a detector, a valve, and a mixer. All of the micro components are in fluid communication with one another. Here, the term “micro” is not limited to a micron or microliter, but may also refer to a nanometer or nanoliter, or millimeter or milliliter.

The biological analysis device for performing a series of sample preprocess operations includes a plurality of chambers. Each chamber includes microfluid that flows inside the chamber and between chambers due to mechanical driving power.

Referring to FIG. 2, the plurality of chambers include a lysis solution chamber 110 for storing a solution which lyses cells in a biological sample so as to extract a desired biomolecule from the biological sample injected into the biological analysis device, a binding solution chamber 120 which is connected to the lysis solution chamber 110 for storing a solution which binds the biomolecule discharged from the lysed cell to a solid material, a washing solution chamber 130 for storing a solution which washes and removes materials that are not bound to the solid material, excluding the biomolecule to be analyzed, and an eluate solution chamber 140 for storing a solution containing the biomolecule eluted from the solid material. A chamber for solid material collection 150 is connected to a binding solution chamber 120 containing a solid material and may receive the solid material from the chamber 120 and a chamber for storing an elution solution 160 is connected to the chamber 150 from the upstream side and waste chamber 170 is connected to the chamber 150 from the downstream side and stores the wastes. A filter 180 is located in fluid path between the chamber 150 and waste chamber 170 or eluate chamber 140. Each chamber is connected to the other chambers by a channel that acts as a fluid path between the chambers, and valves V1, V2, V3, V4, V5, V6, and V7 adjust opening/closing of the corresponding channels.

Some chambers need to be heated so as to efficiently perform molecule extraction processes according to a series of reactions in each chamber. The lysis solution chamber 110 may be heated to heat the lysis solution therein, and the heated lysis solution may be used to efficiently lyse and disrupt cell membranes in the biological sample by contacting the heated lysis solution with the cells. The elution solution chamber 160 may be heated to heat the elution solution therein, and the heated elution solution may be used to efficiently separate the biomolecule from the solid material by contacting the heated elution solution to the solid material bound with a biomolecule in the chamber 150. The solid material may be any solid material known to bind a biomolecule such as nucleic acid, protein, or a carbohydrate. The solid material includes silica-based materials known to bind nucleic acid. The solid material may be a solid material contained in a commercially available biomolecule extraction kit such as a nucleic acid, protein and/or carbohydrate extraction kit, for example, from QIAGEN, INVITROGEN Inc, and the like. The heated lysis solution and elution solution may be transported to the lysis chamber and/or elution chamber containing the solid material bound with a biomolecule such as nucleic acid, protein, and carbohydrate. However, the binding solution chamber 120 and the washing solution chamber 130 may not be heated, because when they are heated, the biomolecule may be lost. Accordingly, only some of the chambers are heated.

According to an embodiment, a desired chamber is selectively and quickly heated by adding anions having high charge density that accelerate polarization of the molecules of the liquid medium, thereby reducing difficulties of organizing and controlling a system caused by a resistance heating method and reducing the heating time.

The adding amount of the anions having high charge density is not limited, and is controlled according to types of a cell and a biomolecule, concentration of a biological sample, and type of used anions, while performing desired cell lysing and biomolecule elution processes.

However, when the adding amount of the anions is too small, the heating rate may not reach expectations, and when the adding amount of the anions is excessive, stability may deteriorate as it may be difficult to use extracted biomolecules due to excessive evaporation of the biological sample. Accordingly, the anions may be added in amounts of about 10 mM to about 10 M, specifically in amounts of about 100 mM to about 3M.

The anions may be added in an ionic salt form via an inlet port of each chamber, and the mobility of the anions is controlled by the valves V1 through V7 that control mobility of fluid between the chambers.

The biomolecule obtained through such molecule extraction processes is later detected and analyzed after being amplified through a process such as PCR.

Aside from the use in separating and purifying desired nucleic acid molecules by selectively heating the chambers as described above, the heating method, which is used to selectively and quickly heat required sections of the biological analysis device, may be used during an inactivation process that cleaves proteins and peptides (by using protease enzymes) into fragments, a denaturation process of enzyme or protein/peptide, a processing or incubating process of a protein-protein complex, a nucleic acid-protein complex, a nucleic acid-nucleic acid complex, or a complex of one of the biomolecules and a drug or organic/inorganic compound.

According to an embodiment, there is provided a biological analysis device containing anions, the biological analysis device including a plurality of chambers for processing, removing, or reacting a sample; a liquid medium contained in the plurality of chambers, wherein the liquid medium includes molecules that undergo hydrogen bonding with one another; the anions having a high charge density, the anions being added to some of the plurality of chambers and interacting with the molecules of the liquid medium with a force that is stronger than the forces that produce hydrogen bonding between the molecules of the medium.

As described above, the biological analysis device is a device for detecting and analyzing a biomolecule, and includes a plurality of chambers for performing a series of operations to extract a biomolecule. The anions having high charge density are contained in some of the plurality of chambers. Accordingly, desired chambers are quickly heated by selectively applying microwaves thereto.

The anions having high charge density that accelerates polarization of molecules of the liquid medium may be added to chambers that need to be heated, such as the lysis solution chamber 110 and/or the elution solution chamber 160 of FIG. 2. The anions having high charge density may be one of citric acid ions (citrate³), sulfate ions (SO₄²⁻), hydrogen sulfate ions (HSO₄⁻), phosphate ions (PO₄³⁻), hydrogen phosphate ions (HPO₄²⁻), carbonate ions (CO₃²⁻), hydrogen carbonate ions (HCO₃⁻), or a combination comprising at least one of the foregoing ions.

Meanwhile, anions having low charge density may be added to chambers that do not need to be heated. For example, anions having low charge density may be directly added to the binding solution chamber 120 and the washing solution chamber 130 of FIG. 2 in an ionic salt form, so as to prevent deterioration of a molecule recovery rate. The anions having low charge density may be one of acetic acid ions (acetate⁻), chloride ions (Cl⁻), nitrate ions (NO₃⁻), bromide ions (Br⁻), chloric ions (ClO₃⁻), iodides (I⁻), perchlorate ions (ClO₄⁻), thiocyanate ions (SCN⁻), or a combination comprising at least one of the foregoing low charge density anions.

According to an embodiment, there is provided a dielectric substance for dielectric heating, the dielectric substance including a medium that emits heat as a hydrogen bond between medium molecules is weakened by microwaves, and anions having high charge density that interact with the molecules of the liquid medium. As noted above, the anions being added interact with the molecules of the liquid medium with a force that is stronger than the forces that produce hydrogen bonding between the molecules of the medium. The medium may be water, an aqueous solution, organic solvent, or a buffer solution.

### Example 1

### 1-1. Preparing Anions Having High Charge Density

Salts (sodium carbonate, sodium phosphate (dibasic), sodium sulfate, and sodium citrate) including anions having high charge density were each dissolved in distilled water in a concentration of 100 millimolar (“mM”) to prepare aqueous solutions. Then, 1 milliliter (“ml”) of each aqueous solution was separately put into a micro tube.

### 1-2. Irradiating Microwave

The aqueous solutions were irradiated with microwaves including the anions having high charge density prepared in 1-1 above for 10 seconds in a commercial microwave oven. The microwaves have a frequency of 2.45 GHz.

### Comparative Example 1

1-1 and 1-2 were performed in the same manner as Example 1, except that 1 ml aqueous solutions were prepared by mixing distilled water with each of sodium chloride, sodium acetate, and N-cyclohexyl-3-aminopropanesulfonic acid (“CAPS”) (as zwitterions) to have a concentration of 100 mM.

### Experimental Example 1

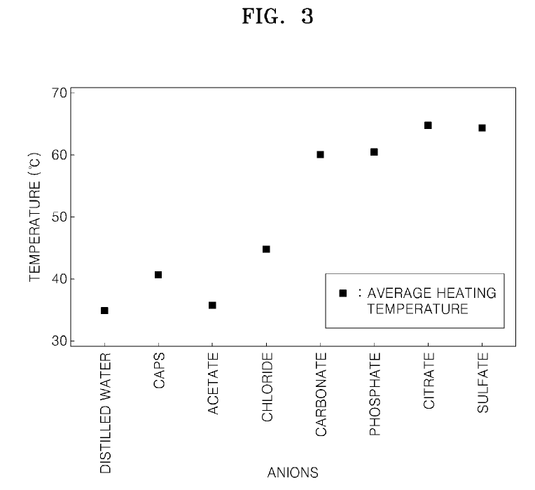

Average temperatures and heating rates according to dielectric heating in Example 1 and Comparative Example 1 were measured, and the results are shown in Table 1 and FIG. 3.

TABLE 1

| | Type of | Average | Heating Rate |
|---|---|---|---|
| | Aqueous Solution | Temperature (° C.) | (° C./sec) |
| Example 1 | Sodium Citrate | 64.7 | 6.5 |
| | Sodium Sulfate | 64.3 | 6.4 |
| | Sodium Phosphate | 60.3 | 6.0 |
| | (Dibasic) | | |
| | Sodium Carbonate | 60.0 | 6.0 |
| Comparative | Sodium Chloride | 44.7 | 4.5 |
| Example 1 | Sodium Acetate | 36.0 | 3.6 |
| | CAPS | 40.7 | 4.1 |
| | Distilled Water | 35.0 | 3.5 |

Referring to Table 1 and FIG. 3, the aqueous solutions including the anions having high charge density, i.e. the aqueous solutions including sodium citrate, sodium sulfate, sodium phosphate (dibasic), and sodium carbonate, were quickly heated in 10 seconds to a temperature that is about 25° C. higher than the aqueous solutions including sodium chloride and sodium acetate, which are ionic salts having low charge density, and CAPS, which is a zwitterionic compound. Zwitterionic compounds include the same amount of positive and negative charges. Also, the aqueous solutions of Example 1 were heated about 30° C. higher than distilled water.

### Example 2

1-1 and 1-2 were performed in the same manner as Example 1, except that 1 ml aqueous solutions were prepared by mixing distilled water with each of sodium phosphate (monobasic), sodium phosphate (dibasic), and sodium phosphate (tribasic) to have a concentration of 100 mM.

### Experimental Example 2

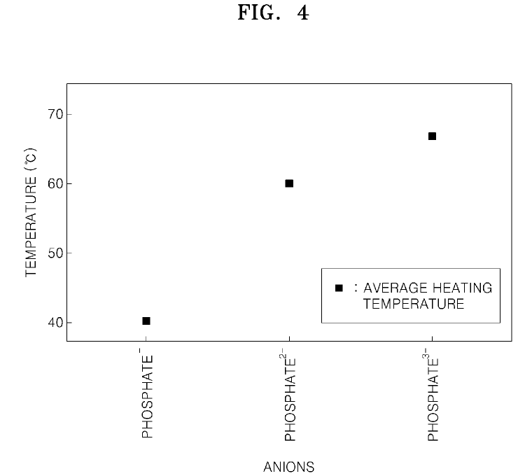

Average temperatures and heating rates of the aqueous solutions prepared in Example 2 according to dielectric heating were measured, and the results are shown in Table 2 and FIG. 4.

TABLE 2

| | | Average | |
|---|---|---|---|
| | | Temperature | Heating Rate |
| | Type of Aqueous Solution | (° C.) | (° C./sec) |
| Example 2 | Sodium Phosphate (Tribasic) | 67.0 | 6.7 |
| | Sodium Phosphate (Dibasic) | 60.3 | 6.0 |
| | Sodium Phosphate (monobasic) | 40.3 | 4.0 |

Referring to Table 2 and FIG. 4, sodium phosphates, which are salts including phosphate ions as anions having high charge density, were heated. Tribasic, dibasic, and monobasic sodium phosphates were used. As a result, as the ion charge density increased, heating rate was increased.

### Example 3

1-1 and 1-2 were performed in the same manner as Example 1, except that sodium dodecyl sulfates (“SDSs”) were prepared in various concentrations in 1-1 as shown in Table 3 below.

### Comparative Example 2

1-1 and 1-2 were performed in the same manner as Example 1, except that 1 ml distilled water was prepared in 1-1.

### Experimental Example 3

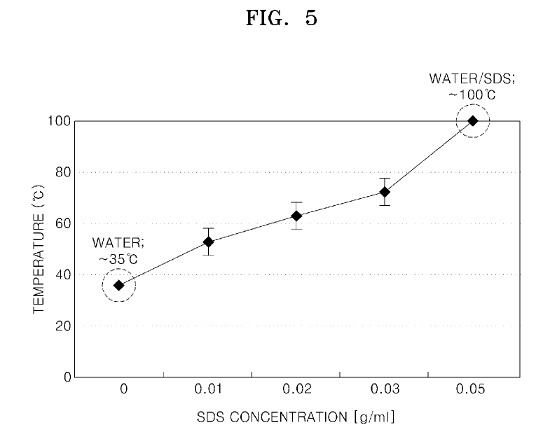

Average temperatures according to dielectric heating performed in Example 3 and Comparative Example 2 were measured, and results are shown in Table 3 and FIG. 5.

TABLE 3

| | Type of Aqueous | Average | Heating |
|---|---|---|---|
| | Solution | Temperature (° C.) | Rate (° C./sec) |
| Example 3 | SDS 0.01 g/ml | 50.0 | 5.0 |
| | SDS 0.02 g/ml | 61.0 | 6.1 |
| | SDS 0.03 g/ml | 70.0 | 7.0 |
| | SDS 0.05 g/ml | 100.0 | 10.0 |
| Comparative | Distilled Water | 35.0 | 3.5 |
| Example 2 | | | |

Referring to Table 3 and FIG. 5, SDSs including anions having high charge density were heated while differentiating the concentration from 0.01 to 0.05 g/ml, and the heating rate increased as the concentration increased. Specifically, in the case of 0.05 g/ml, the heating temperature increased up to 100° C. in 10 seconds.

In other words, by suitably adjusting the concentration of anions having high charge density and the time of irradiating the microwaves, heating may be performed selectively within a short time.

As described above, according to the one or more of the above embodiments, a heating method using microwaves quickly and selectively heats only desired substances by adding anions having high charge density that accelerate polarization of molecules in the desired dielectric substances.

Also, while heating a biological analysis device including a plurality of chambers containing a fluid, only desired chambers are selectively and quickly heated.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed is:

1. A method comprising:
adding anions having high charge density to a liquid medium; the liquid medium comprising molecules that hydrogen bond with one another; the anions interacting with the molecules of the liquid medium with a force that is stronger than the forces that produce hydrogen bonding between the molecules of the medium; and
heating the liquid medium by irradiating it with microwaves.

2. The method of claim 1, wherein the liquid medium comprises any one of water, an aqueous solution, organic solvent, a buffer solution, or a combination thereof.

3. The method of claim 1, wherein the anions having a high charge density are selected from the group consisting of citric acid ions (citrate³⁻), sulfate ions (SO₄²⁻), hydrogen sulfate ions (HSO₄⁻), phosphate ions (PO₄³⁻), hydrogen phosphate ions (HPO₄²⁻), carbonate ions (CO₃²⁻), hydrogen carbonate ions (HCO₃⁻), and a combination comprising at least one of the foregoing anions having a high charge density.

4. The method of claim 1, wherein the anions having high charge density are added in a concentration of about 100 mM to about 3 M.

5. The method of claim 1, wherein the microwaves have a frequency of about 300 MHz to about 30 GHz.

6. A method of heating a liquid medium comprising:
disposing a liquid medium in a chamber of a biological analysis device; the liquid medium comprising molecules that hydrogen bond with one another;
adding anions having a high charge density to the chamber; the anions interacting with the molecules of the liquid medium with a force that is stronger than the forces that produce hydrogen bonding between the molecules of the medium; and
heating the chamber by irradiating it with microwaves.

7. The method of claim 6, wherein the chamber is any one of a lysis solution chamber for storing a solution that lyses cells in a biological sample, and a elution solution chamber for storing a solution that elutes a biomolecule from a solid material to which the biomolecule is bound.

8. The method of claim 6, wherein the anions having high charge density are selected from the group consisting of citric acid ions (citrate³⁻), sulfate ions (SO₄²⁻), hydrogen sulfate ions (HSO₄⁻), phosphate ions (PO₄³⁻), hydrogen phosphate ions (HPO₄²⁻), carbonate ions (CO₃²⁻), hydrogen carbonate ions (HCO₃⁻), and a combination comprising at least one of the foregoing anions having a high charge density.

9. The method of claim 6, wherein the anions having a high charge density are added in a concentration of about 100 mM to about 3 M.

10. A biological analysis device comprising:
a plurality of chambers for processing, removing, or reacting a sample;
a liquid medium contained in the plurality of chambers, wherein the liquid medium comprises molecules that hydrogen bond with one another; and
anions having high charge density, wherein the anions are added to some of the plurality of chambers and interact with the molecules of the liquid medium with a force that is stronger than the forces that produce hydrogen bonding between the molecules of the medium.

11. The biological analysis device of claim 10, wherein the medium comprises any one of water, an aqueous solution, organic solvent, a buffer solution, or a combination thereof.

12. The biological analysis device of claim 10, being any one of a microfluidic device, a microfluidic cartridge, a lab-on-a chip, and a lab-on-a disc, for detecting or analyzing a biomolecule from a biological sample.

13. The biological analysis device of claim 10, wherein the chamber is any one of a lysis solution chamber for storing a solution that lyses cells in a biological sample, and a elution solution chamber for storing a solution that elutes a biomolecule from a solid material to which the biomolecule is bound.

14. The biological analysis device of claim 13, wherein the biological sample comprises any one of a cell suspension including microorganisms, blood, urine, or saliva of a living being, and the biomolecule comprises any one of nucleic acid, protein, peptide, antibody, or hormone.

15. The biological analysis device of claim 10, wherein the plurality of chambers comprise:
a lysis solution chamber for storing a lysis solution, which lyses cells in a biological sample;
a binding solution chamber for storing a binding solution, which binds biomolecules, discharged from the lysed cells to a solid material;
a washing solution chamber for storing a washing solution which removes biomolecules that are not bound to the solid material, by washing the solid material; and
an elution solution chamber for storing an elution solution, which elutes the biomolecules from the solid material to which the biomolecules are bound.

16. The biological analysis device of claim 10, wherein the anions having high charge density are added to chambers that are required to be heated from among the plurality of chambers.

17. The biological analysis device of claim 15, wherein the chambers that are required to be heated are a lysis solution chamber and an elution solution chamber.

18. The biological analysis device of claim 10, further comprising anions having low charge density, which are added to chambers that are not required to be heated from amongst the plurality of chambers.

19. The biological analysis device of claim 15, wherein the chambers that are not required to be heated are a binding solution chamber and a washing solution chamber.

20. The biological analysis device of claim 18, wherein the anions having low charge density comprise at least one selected from the group consisting of acetic acid ions (acetate-), chloride ions (Cl⁻), nitrate ions (NO₃⁻), bromide ions (Br⁻), chloric ions ClO₃⁻), perchloric ions (ClO₄⁻), iodides (I⁻), thiocyanate ions (SCN⁻), and combinations thereof.

21. A medium comprising:
a liquid medium; the liquid medium comprises molecules that hydrogen bond with one another; and
anions; the anions having a high charge density; the anions interacting with the molecules of the liquid medium with a force that is stronger than the forces that produce hydrogen bonding between the molecules of the medium.

22. The liquid medium of claim 21, wherein the medium comprises any one of water, an aqueous solution, organic solvent, and a buffer solution.
